(19) United States
(12) Patent Application Publication
(10) Pub. No.: US 2024/0316064 A1
(43) Pub. Date: Sep. 26, 2024

(54) USE OF ESTRETOL AS A TREATMENT FOR ENDOMETRIOSIS

(71) Applicants: UNIVERSIDAD BERNANDO O'HIGGINS, Santiago (CL); PONTIFICIA UNIVERSIDAD CATOLICA DE CHILE, Santiago (CL)

(72) Inventors: Renán ORELLANA WALDEN, Santiago (CL); Ricardo MORENO MAURO, Santiago (CL); Daniel PATIÑO GARCIA, Santiago (CL)

(21) Appl. No.: 18/576,018

(22) PCT Filed: Jun. 28, 2022
(86) PCT No.: PCT/CL2022/050066

§ 371 Date: Mar. 18, 2024

(30) Foreign Application Priority Data
Jul. 1, 2021 (CL) 1762-2021

(51) Int. Cl. A61K 31/57; A61K 31/565; A61P 15/00

(52) U.S. Cl. CPC A61K 31/57; A61K 31/565; A61P 15/00

(57) ABSTRACT

The present invention relates to the use of a combination of estetrol (E4) and progesterone (P4) to prepare a medicine that is useful in the treatment of endometriosis. The medicine of the invention does not lower progesterone-receptor (PR) levels, but does increase progesterone-receptor levels in endometrial cells, causing the disease to progress more slowly and preventing the development of resistance to treatment.

### FIELD OF INVENTION

The invention is directed to a combination of estetrol (E4) and progesterone (P4), and its use to prepare a medicament useful in the treatment of endometriosis.

### BACKGROUND OF THE INVENTION

Endometriosis is a common gynecological disease that affects 6 to 10% of women of childbearing age, causing painful pelvic lesions and infertility. Endometriosis is defined as the presence of endometrial tissue, both stroma and glands, outside the uterus. Where the proliferation, invasion and survival of the lesions is accentuated and/or maintained, due to the effects of estradiol (E2), during each menstrual cycle. On the other hand, progesterone (P4) promotes tissue atrophy, its apoptosis and autophagy. For this reason, current treatments focus on stimulating the response to P4 or lowering the effects of E2. Progestins are widely used since they manage to mitigate a large part of the symptoms (inflammation and volume of the lesions) and also slow their progress by reducing the plasmatic level of E2, through its inactivation to estrone. However, many patients treated with progestins develop hypoestrogenism (a sharp drop in E2 levels) that leads to serious side effects, bone decalcification, and vascular accidents. For this reason, combined progestin and low-dose estrogen therapies have shown efficacy in reducing pain and lowering side effects.

However, one third of patients become insensitive to progesterone (P4), stop responding to treatment and therefore re-develop symptoms of the disease, it is believed that this happens due to a reduction in receptor levels for P4 (PR), which is known as “progesterone resistance”. The only possible solution is surgery, an option that may be necessary on a recurring basis.

Estetrol (E4) is a natural estrogen produced by the liver of the fetus during pregnancy. It is currently in industrial production and study as a contraceptive, hormonal treatment for climacteric and breast cancer.

For example, in the recent publication by Gaspard, Ulysse, et al. (“A multicenter, randomized study to select the minimum effective dose of estetrol (E4) in postmenopausal women (E4Relief): part 1. Vasomotor symptoms and overall safety.” Menopause (New York, NY) 27.8 (2020): 848) evaluates its use to reduce the discomforts of menopause, such as hot flushes.

Another recent publication describes the use of estetrol in contraceptive treatments, Grandi, Giovanni, et al. (“Estetrol (E4): the new estrogenic component of combined oral contraceptives.” Expert review of clinical pharmacology 13.4 (2020): 327-330.) The paper describes the benefits of using estetrol as a contraceptive, in combination with drospirenone (DRSP) and levonorgestrel (LNG). Its use in endometriosis is not anticipated in this review.

Patent application US2021046084A1 “Treatment of advanced estrogen receptor positive breast cancer, 2021-02-18”, points to the use of estetrol as part of a specific treatment in breast cancer.

We have not found in the prior art studies that support that estetrol can be used in the treatment of endometriosis.

The inventors have developed a new hormonal combination, which comprises estetrol and progesterone that allows its application in the treatment of endometriosis, since surprisingly, unlike the estradiol that has been used up to now, estetrol does not generate proliferation or migration of endometriotic cells. and further modulates the levels of receptors in these same cells so that the disease progresses more slowly and does not develop resistance to treatment, as shown later in the examples.

### DESCRIPTION OF THE FIGURES

FIG. 1. Evaluation of apoptosis, cell viability and migration after treatments with E2 and E4 in endometriotic cells. (A) Western blot analysis against PARP and β-actin after 24 h incubation (B) MTS assay after 24 h incubation (C) Scratch assay. Results are shown for cells 11Z (left) and Hs832 (right) (n=6), *p<0.05, ** p<0.01, *** p<0.001, by one-way ANOVA together with Dunnett's post hoc test.

FIG. 2. Measurement of ER protein levels by Western blot after E2 or E4 treatments in endometriotic cells. (A) ERα levels above B-actin (AU) levels for 11Z (left) and Hs832 (right) cells. (B) ER β levels over β-actin (AU) levels for 11Z (left) and Hs832 (right) cells (n=6), (c) the ratio between ERα levels/ERβ levels * p<0.05, ** p<0.01, *** p<0.001, by one-way ANOVA together with Dunnett's post hoc test.

FIG. 3. Measurement of PR protein levels by Western blot after E2 or E4 treatments in endometriotic cells. (A) PRA levels above β-actin (AU) levels for 11Z (left) and Hs832 (right) cells. (B) PRB levels over β-actin (AU) levels for 11Z (left) and Hs832 (right) cells (n=6), *p<0.05, ** p<0.01, *** p<0.001, by one-way ANOVA together with Dunnett's post hoc test.

FIG. 4. PCR Array Analysis for Progesterone-Regulated Genes in Endometrial Cells, Hs832 Cells. The expression of genes regulated by progesterone (P4) is compared, when incubating with P4 without previous treatment or with a 24 hour treatment with Estetrol. It is observed that the expression differs in both cases, stimulating the expression of NFIX, GNRFI1, ESR1, MEFD2 and I HH, to a lesser extent they increase the expression of PTGER2, NF1, MUC1, TWIST1 and SNAI1. And strongly decreasing the expression of PRL, SNAI2, RAD54L, HES1, ABCG2, TCF4, and to a lesser extent FGF12, GCFC2, PWP1, ITGA6, ZEB1, POSTN and FKBP25

### DESCRIPTION OF THE INVENTION

The invention is aimed at an effective treatment of endometriosis, wherein a composition comprising a combination of estetrol and progesterone is administered, together with pharmaceutically acceptable excipients and carriers. Considering the clinical studies to date, this combination can be administered orally, so it can be formulated as dragees or tablets.

Thus, the invention consists of a pharmaceutical composition for the treatment of endometriosis comprising a combination of estetrol (E4) and progesterone (P4), pharmaceutically suitable excipients and carriers, where estetrol (E4) and progesterone (P4), are in a 1:10 to 1:100 ratio, especially a 1:10 ratio. Where these components reach a concentration of 10⁻⁹ to 10⁻⁵M for estetrol (E4) and 10⁻⁸ to 10⁻⁶M for progesterone (P4).

This composition makes it possible to prepare a medicament useful for the treatment of endometriosis, where the medicament does not generate a reduction in the levels of progesterone receptors (PR), but even increases the levels of progesterone receptors in endometriotic cells.

The medicament can be formulated for oral administration, either as simple lozenges or tablets or within a controlled release formulation.

The drug formulation comprises between 1 and 50 mg of estetrol and between 50 and 200 mg of progesterone.

The medicine can be used daily for at least 1 month, and for as long as the doctor determines, which can be throughout the patient's fertile life.

As indicated, this composition is advantageous in the preparation of a drug useful for the treatment of endometriosis, where this drug generates a reduction in the levels of estrogen receptors (ER), and at the same time increases the levels of progesterone receptors in endometriotic cells, that is, contrary to what happens with estradiol, resistance to progesterone would not be generated. In addition, by not inducing cell proliferation or migration, the effectiveness of the composition would increase. This particularity of the medicine of the invention has a high impact on the patients who will use it, since the disease can be continuously controlled by the composition of the invention and the need for surgery is reduced.

Estetrol (E4), a natural estrogen with low side effects, currently in clinical trials for hormone treatment in women, has preferential affinity for ERα. The inventors evaluated the effects of E4 on endometriotic cells. Epithelial (11Z) and stromal (Hs832) endometriotic cell lines were incubated with E4 and compared with 17-B estradiol (E2), evaluating critical events for endometriosis such as proliferation, migration, and levels of estrogen and progesterone receptors. The results show that E4 does not increase the factors that favor the disease.

The inventors have studied the interaction of estetrol with endometrial cells, finding, firstly, that estetrol does not modify the viability of cells compared to the control and secondly does not induce cell migration, in contrast to what occurs with estradiol. Where this last compound increases the number of endometriotic cells and induces their migration. This result is very positive given that one of the characteristics of the disease is that endometriotic cells invade other tissues, migrating and proliferating, which would be favored by estradiol, but surprisingly, not by estetrol.

It is known that the estrogen response is a crucial event for the progression of endometriosis, and this response is determined on the one hand by the E2 concentration, and at the same time by the levels of the different estrogen receptors in the endometrial cells. A significant increase in ERα levels or reduction of ERβ can be considered as a beneficial effect for the treatment of endometriosis. It has been established that the ERα/ERβ ratio is chronically reduced in endometriosis. The inventors studied the effect of estetrol in the presence of these receptors, and found that, surprisingly, treatment of cells with estetrol raises the ERα/ERβ ratio, as shown in the examples.

Thus, estetrol itself has effects that contribute to diminishing endometriosis, furthermore, enhancing the progesterone response. Additionally, the inventors evaluated the response of endometrial cells to progesterone in the presence and absence of estetrol, and observed that there are significant differences between both conditions.

Thus, the invention provides a new drug for the treatment of endometriosis, which would replace the current combination of estradiol and progesterone with the new combination of estetrol and progesterone, which would be significantly more effective. Where estetrol contributes to progesterone in the reduction of the disease, contrary to what happens with the current treatment, where the estradiol maintains the pathology.

It should be considered that both estetrol and progesterone are currently approved as drugs and are used for different purposes. Progesterone combined with estradiol for endometriosis, and sterol as drugs to reduce bothersome symptoms of menopause, for example.

The examples included below show the effects of the components of the invention in an in vitro model, such as endometrial cell lines. It will be evident to the person skilled in the art that the behavior of these cells predicts the in vivo behavior of the tissue. These examples are to be understood as illustrative and not limiting of the scope of the invention.

### EXAMPLES

### Example 1. E4 Reduces Apoptosis and does not Affect Cell Number or Migration in Endometriotic Cells

The experiments were performed on 2 cell lines, Hs832, endometrial stromal cells and 11Z cells, endometrial epithelium cells.

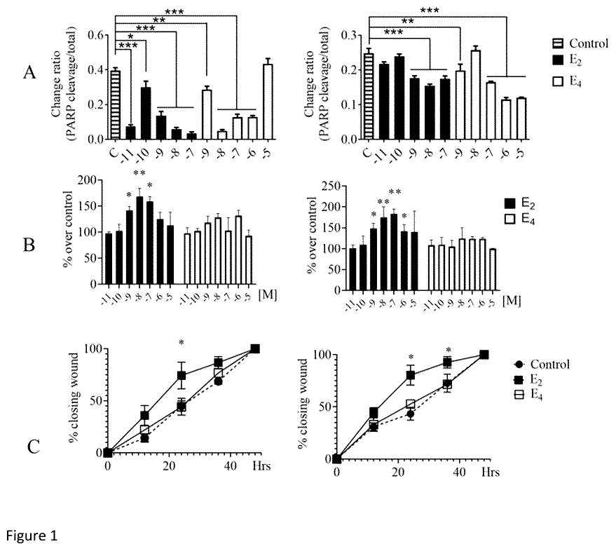

Firstly, the PARP cleavage rate was evaluated as a measure of apoptosis, the cell lines Hs832 and 11Z were incubated at physiological and supraphysiological concentrations of estradiol and estetrol separately, where the physiological concentrations of estradiol was 10 8M and estetrol was 10⁻⁶M. Both hormones showed a similar and consistent pattern of reduced PARP cleavage rate. For 11Z epithelial cells, apoptosis was reduced at most estetrol and estradiol concentrations (FIG. 1A, left). For the Hs832 stromal cell line, a significant decrease was only observed for two conditions (10⁻⁹/10⁻⁷M) of estradiol and almost all estetrol concentrations (except 108M) (FIG. 1A, right).

To assess cell viability, the MTS assay was performed after 24 h. Incubation of 11Z epithelial cells with 109 to 10⁻⁷M estradiol showed a strong increase, from 41% to 68% over the control (FIG. 1B, left). The Hs832 stromal cell line incubated with 109 to 10⁻⁶M estradiol showed a 41% up to 83% increase over the controls (FIG. 1B, right). None of the estetrol concentrations tested showed changes in cell viability for both cell lines, compared to controls.

Migration, a crucial step for injury establishment, was assessed by a wound assay with estradiol and estetrol at physiological concentrations (10⁻⁹ and 10⁻⁶ respectively, FIG. 1C). For both cell lines, estradiol significantly increases migration (11Z: 24 h and Hs832: 24 h and 36 h, FIG. 1C). Again, estetrol showed no difference from the control at any time point studied.

ESTETROL increases the ERα/ERβ ratio according to your protein levels.

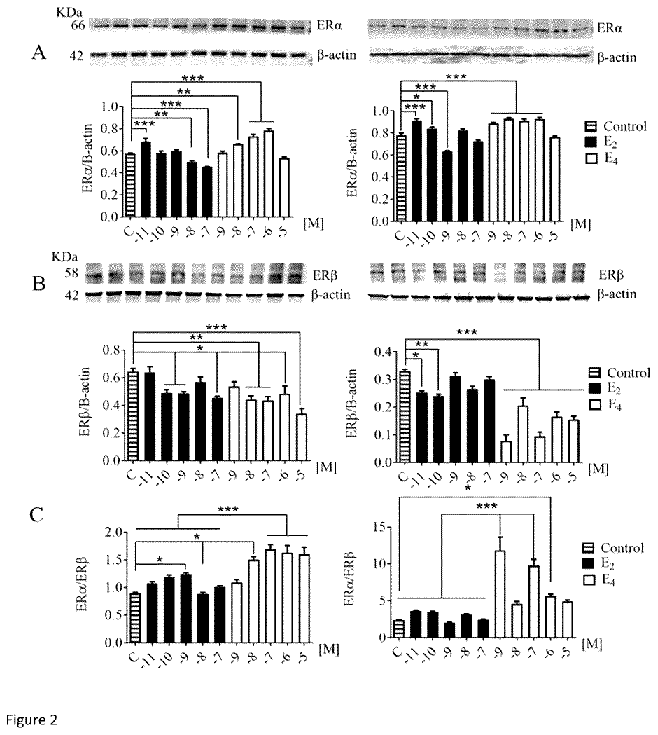

Considering the role of ERs in endometriosis, we evaluated their levels after estradiol or estetrol incubation in epithelial and stromal cells. For ERα, ESTRADIOL administration resulted in propagation results showing increased (11Z: 10⁻¹¹M; Hs832: 10⁻¹¹/10⁻¹⁰M, FIG. 2A) and decreased levels in both cell lines (11Z: 10⁻⁸/10⁻⁷M; Hs832: 10⁻⁹M, FIG. 2A). 2A). Interestingly, incubation with ESTETROL showed a robust increase in ERα levels in both cell lines at most of the concentrations used (FIG. 2B). Regarding ER levels, there was a significant decrease after incubation with estradiol in some conditions (11Z: 10⁻¹⁰/10⁻⁹/10⁻⁷M; Hs832: 1011/1010M, FIG. 2B). For estetrol, reduced levels of ER protein were observed at most concentrations used for 11Z and all conditions for Hs832 cells (FIG. 2B). Finally, in FIG. 2C, estetrol significantly increases the ERα/ERβ ratio with respect to the control and especially with respect to the estradiol conditions, this was observed at 10⁻⁷/10⁻⁶/10⁻⁵M for 11Z cells and at 10⁻⁹/10⁻⁷M for Hs832 cells. For estradiol, the ERα/ERβ ratio increased only in epithelial cells at 10⁻⁷M.

ESTETROL increases PR protein levels.

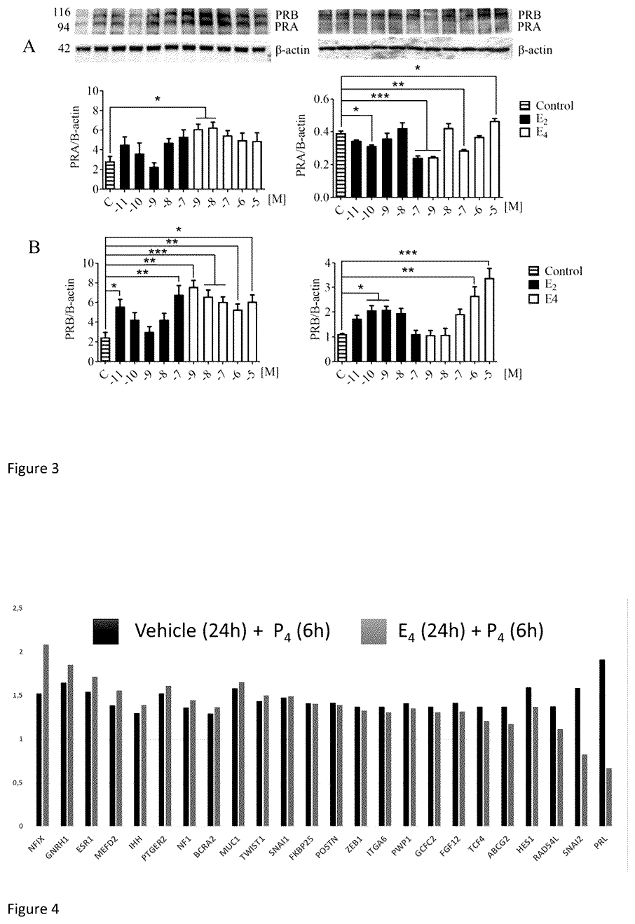

Evaluation of PR progesterone receptor protein levels showed an extended response to PRA depending on conditions and cell lines. Incubation with estradiol alone showed a significant decrease in protein levels for Hs832 cells at 10⁻¹⁰/10⁻⁷M. In the case of incubation with estetrol, it induced an increase in PRA protein levels (11Z: 10⁻⁹/10⁻⁸M; Hs832: 10⁻⁵M, FIG. 3A), but it also decreased two conditions (HsS32: 10⁻⁹/10⁻⁷M, FIG. 3A). For PRB protein levels, incubation with estradiol resulted in increased levels at some concentrations (11Z: 10⁻¹¹/10⁻⁷M: HsS32: 10⁻¹⁰/10⁻⁹M, FIG. 3B). For estetrol, a more consistent increase was observed for all concentrations used in 11Z cells and in 10⁻⁶/10⁻⁵M for HsS32.

### Example 2. Expression Analysis of Genes Regulated by Progesterone in the Presence of Estetrol

Procedure: Hs832 endometriosis cells were incubated for 24 h with E4 (10⁻⁶M) or vehicle (ethanol) as a control, then both cells received a 6h pulse with P4 (10⁻⁷M) to then extract their messenger RNA in order to evaluate the levels. expression of 24 genes associated with the progesterone signaling pathway.

Pre-treatment with E4 modifies the response to progesterone in a variety of ways, observing genes that increase, decrease or do not change their expression levels. This change in the expression pattern may be associated with an effect on PR receptor levels (FIG. 4). It is observed that the expression differs in both cases, stimulating the expression of NFIX, GNRFI1, ESR1, MEF2D and I HH, to a lesser extent the expression of PTGER2, NF1, MUC1, TWIST1 and SNAI1 increases. Where all these genes are associated with appropriate physiological cellular processes related to programmed cell death. Likewise, it is observed that treatment with E4 strongly decreases the expression of PRL, SNAI2, RAD54L, HES1, ABCG2, TCF4, and to a lesser extent FGF12, GCFC2, PWP1, ITGA6, ZEB1, POSTN and FKBP25. Where all these genes are associated with pathological processes, specifically with different stages of cancer.

This gene ontology analysis showed that incubation with estetrol potentiates processes associated with apoptosis and cell death, through the activation of specific genes of the progesterone pathway. On the other hand, an ontological analysis focused on pathological processes, using genes decreased by estetrol treatment, showed that among the 10 main molecular events involved, 5 are related to cancer processes and cell malignancy (invasion, migration, survival, among others), which are important for the development of endometriotic lesions of the disease. The summary of this analysis and of the processes modulated by estetrol are summarized in Table 1.

Therefore, the estetrol and progesterone treatment of the invention, which obviously includes estetrol, is more effective in inducing endometriotic cell death, while that reduces their invasive capacity, both conditions are favorable for the treatment and reduction of the endometriosis disease.

TABLE 1
Gene Ontology results. Left column: “cellular processes”
analysis for genes upregulated by estetrol, performed in “g:Profiler”
of “ELIXIR infrastructure”. Right column: “pathology”
analysis for estetrol-depleted genes, performed in ToppGene Suite.

| Cellular processes | Pathological processes |
|---|---|
| increased by E4 | decreased by E4 |
| Apoptosis process | Recurrent tumors |
| Programmed cell death | Tumor initiation |
| Cell death | Basal type breast carcinoma |
| Regulation of apoptosis processes | Esophageal neoplasm |
| Regulation of programmed cell death | Malignant esophageal neoplasm |

### Example 3. Pharmaceutical Formulation

A dose of between 0.05 to 0.5 mg/Kg of weight of the patient of estetrol reaches an availability within the nanomolar range (109, 108 and 107M) at which we observed the beneficial effects for the treatment of endometriosis.

The formulation consists of a tablet obtained by compression of a dry composition of the following compounds:
15 mg estetrol
150 mg progesterone
145 mg of excipients (30% corn starch and 70% Microcrystalline Cellulose type PH 102) pharmaceutical grade.

Values are expressed per tablet.

These examples should be considered as illustrative and not limiting of the scope of the present invention.

1. Pharmaceutical composition for the treatment of endometriosis wherein it comprises a combination of estetrol (E4) and progesterone (P4), pharmaceutically suitable excipients and carriers.

2. Pharmaceutical composition according to claim 1 wherein it comprises estetrol (E4) and progesterone (P4), in a ratio of 1:10 to 1:100.

3. Pharmaceutical composition according to claim 2 wherein it comprises estetrol (E4) and progesterone (P4), in a ratio of 1:10.

4. Method for the treatment of endometriosis wherein of the composition defined in claim 1 wherein it serves to prepare a useful medication.

5. Use according to claim 4 wherein the medication does not generate a reduction in the levels of progesterone receptors (PR).

6. Use according to claim 5 wherein it increases the levels of progesterone receptors in endometriotic cells.

7. Use according to claim 4 wherein the medicine is provided as dragees or tablets for oral administration.

8. Use according to claim 4 wherein the medication comprises between 1 to 50 mg of estetrol and between 50 to 200 mg of progesterone.

9. Use according to claim 8 wherein the medication is administered daily for at least 1 month.

10. Use according to claim 8 wherein the medication is administered in a controlled release formulation.
